(19) United States
(12) Patent Application Publication
(10) Pub. No.: US 2003/0235903 A1
(43) Pub. Date: Dec. 25, 2003

(54) Propagation of cryptosporidium in cell culture

(75) Inventors: R. C. Andrew Thompson, Roleystone (AU); Nawal S. Hijjawi, Rivervale (AU); Manual Campos, Redwood Meadows (CA); Amber Appelbee, Calgary (CA); Merle E. Olson, Calgary (CA)

Correspondence Address: BURNS DOANE SWECKER & MATHIS L L P, POST OFFICE BOX 1404, ALEXANDRIA, VA, 22313-1404, US

(73) Assignees: University Technologies International, Inc., Calgary (CA); Murdoch University, Murdoch (AU)

(21) Appl. No.: 10/177,251

(22) Filed: Jun. 20, 2002

(51) Int. Cl. C12N 1/10; C07K 16/20

(52) U.S. Cl. 435/258.1; 530/389.5

(57) ABSTRACT

The invention provides an improved method of propagating Cryptosporidium in cell culture. The methods supports the complete life cycle or Cryptosporidium and produces all known forms of the parasite in addition to two novel forms. Cryptosporidium parasites propagated in vitro can be used to produce a vaccine composition for immunizing animals against Cryptosporidium infection.

### FIELD OF THE INVENTION

The present invention relates to the propagation of the intestinal protozoan, Cryptosporidium, in cell culture and the use of Cryptosporidium so prepared in vaccines to prevent or treat human and animal diseases associated with Cryptosporidium.

### BACKGROUND OF THE INVENTION

Parasitic Intestinal Protozoa

Intestinal protozoa cause a variety of clinically and economically important diseases in humans and animals. Examples of known pathogenic intestinal protozoa include Giardia, trichomonads, Histomonas, Spironucleus, Entamoeba, Coccidia, Sarcocystis, Toxoplasma, and Cryptosporidium.

Cryptosporidium is an Apicomplexan protozoan parasite that invades the intestinal epithelial cells of various mammals, including humans, domesticated farm animals, and poultry. In humans, the parasite infects the microvillis border of the intestinal epithelium, causing acute, self-limiting diarrhea in immunocompetent individuals, and a chronic, life-threatening disease in immunocompromised patients. C. parvum demonstrates broad mammalian host specificity, infecting humans through direct human contact and via zoonotic transmission and has become a leading cause of diarrhea in calves. C. baileyi and C. muris have greater specificity for chickens and mice, respectively. [Laurent, F. et al. (1999) Pathogenesis of Cryptosporidium parvum infection. Microbes and Infection. 2:141-148; Gasser, R. B. and O'Donoghue, P. (1999) Isolation, propagation and characteriation of Cryptosporidium. Int. J. Parasitology. 29:1379-1413].

At least two species of Cryptosporidium infect cattle. C. parvum is characterized by small-type oocysts (5.0×4.5 mm) and primarily infects the intestine of young calves resulting in considerable economic losses in the cattle industry and water-borne outbreaks of diarrheal disease in human populations [O'Donoghue, P. J. (1995) Cryptosporidium and cryptosporidiosis in man and animals. Int. J. Parasitol. 25:139-95]. C. andersoni is a recently renamed species characterized by larger oocysts (7.4×5.6 mm) that infects the abomasum (fourth division of the stomach in ruminant animals) of cattle (Lindsay, D. S. et al. (2000) Cryptosporidium andersoni n. sp. (Apicomplexa: Cryptosporiidae) from cattle, Bos taurus. J. Eukaryot. Microbiol. 47:91-95]. The life cycle and propagation of Cryptosporidium are discussed below.

Cryptosporidium Life Cycle and Propagation

Cryptosporidium oocysts are transmitted by the fecal-oral route, particularly though contaminated water supplies and public swimming pools in endemic regions. Following ingestion by a suitable host, Cryptosporidium oocysts excyst in the presence of host bile salts and pancreatic enzymes. The resulting sporozoites infect intestinal epithelial cells, and differentiate into trophozoites. The trophozoites multiply asexually to produce type 1 schizonts containing about 6-8 merozoites. These merozoites can invade additional cells upon rupture of the schizonts. Merozoites may may continue to develop into type 1 schizonts or form type II schizonts which further differentiate into either male microgamonts or female macrogamonts. Male microgamonts release microgametes that fertilize the female macrogamont, resulting in an oocyst. Thick-walled oocysts pass through the digestive tract of the host while thin-walled oocysts likely reinfect the host [Laurent, F. et al. (1999) Pathogenesis of Cryptosporidium parvum infection. Microbes and Infection. 2:141-148; Gasser, R. B. and O'Donoghue, P. (1999) Int. J. Parasitology. 29:1379-1413].

Oocysts and sporozoites can be obtained from infected animals (e.g., calves) in large quantities; however, the handling and maintenance of infected animals constitutes a risk of infection to humans. In addition, obtaining parasites from infected animals presents difficulties in terms of standardization of assays and experimental reproducibility.

Accordingly, practitioners in the field of Cryptosporidium research have attempted to propagate the parasite in vitro. While C. parvum was first grown in culture in 1984 [Current, W. L. and Hayes, T. B. (1984) Complete development of Cryptosporidium in cell cultures. Science. 224:603-05], infections could not be maintained for more than a few days and only the asexual phase of the parasite life cycle was observed. Improvements in Cryptosporidium cell culture methodologies, including the use of a human ileocecal adenocarcinoma cell line (HCT-8), have allowed the study of both the asexual and sexual phase of the Cryptosporidium life cycle; however, infectious oocyst production has remained low [Upton, S. J. et al. (1994) Comparative development of C. parvum. (Apicomplexia) in 11 host cell lines. FEMS Microbiol. Lett. 118:233-36 and Upton, S. J. et al. (1995) Effects of select media supplements on in vitro development of Cryptosporidium parvum in HCT-8 cells. J. Clin. Microbiology 33:371-75].

The ability to propagate Cryptosporidium is essential for understanding host immune response to Cryptosporidium and for developing effective vaccines against Cryptosporidium. IgM, IgG, and IgA antibodies are detectable in the sera of Cryptosporidium-infected animals. IgA may also be present in the intestine. The prevalence of persistent Cryptosporidium infection in AIDS patients, with compromised cellular immunity but substantially normal humoral immunity, suggests that serum antibodies are not protective against Cryptosporidium. However, the production of IgA has been associated with clearance of the parasite in animals. Accordingly, it has been suggested that extracellular forms of Cryptosporidium will be the most plausible targets for protective antibodies [reviewed in Heyworth, M. F. (1992) Immunology of Giardia and Cryptosporidium infections. J. Infect. Diseases 166:465-72 and de Graaf, D.C. et al. (1999) Speculation on whether a vaccine against cryptosporidiosis is a reality or fantasy. Int. J. Parasitol. 29:1289-1306].

The instant invention addresses a need in the art for improved methods of propagating Cryptosporidium in vitro for basic research and for the development of animal and human vaccines.

### SUMMARY OF THE INVENTION

The invention is drawn to an improved method of propagating Cryptosporidium in cell culture based on the diligent maintenance of the pH of the cell culture medium and preventing host cell overgrowth. The method allows the long-term propagation of Cryptosporidium in vitro and supports the complete life cycle of the parasite. The improved cell culture method of the instant invention has further allowed the identification of two extracellular forms of Cryptosporidium that have not previously been observed.

Accordingly, in one aspect the invention provides a method of propagating Cryptosporidium oocysts in vitro comprising the steps of:

a) isolating Cryptosporidium from an infected host organism,

b) excysting the isolated oocysts,

c) recovering the excysted oocysts by centrifugation,

d) resuspending the recovered oocysts in cell culture medium, said medium having a pH within a range of about 7.2-7.6,

e) inoculating cultured mammalian cells with the resuspended oocysts,

f) maintaining the culture of inoculated cells within a pH range of about 7.2-7.6,

g) harvesting supernatant from the inoculated cells, and

h) recovering the oocysts produced in the inoculated cells.

In one embodiment of the invention, Cryptosporidium for propagation in culture are obtained from a host organism selected from the group consisting of a mouse, a human, a bovine, and a pig. In another embodiment of the invention, the Cryptosporidium is selected from the group consisting of Cryptosporidium parvum and Cryptosporidium andersoni.

In another embodiment of the invention, isolated oocysts for propagation in culture are excysted by pretreatment with a trypsin solution at low pH. In a preferred embodiment, the pretreatment solution comprises about 0.5% trypsin and the pH of the pretreatment solution is within a range of about 2.5 to about 3. In a most preferred embodiment, the oocysts are incubated in the pretreatment solution for about 20 minutes at about 37° C.

In one embodiment of the invention, the Cryptosporidium is grown in human adenocarcinoma cells. In a preferred embodiment, Cryptosporidium is grown in HCT-8 cells.

In one embodiment of the invention, the cell culture medium is supplemented with bile. In a preferred embodiment, the concentration of bile is about 0.2 g/L.

In another embodiment of the invention, the cell culture medium is supplemented with an antibiotic or antifungal agent. In a preferred embodiment, the cell culture medium further comprises at least one additional supplement selected from the group consisting of folic acid, 4-aminobenzoic acid, calcium pantothenate, and ascorbic acid.

In another embodiment of the invention, the pH of the cell culture medium is maintained within the range of about 7.2-7.6 by periodically changing the cell culture media. In a preferred embodiment, the medium is changed about every 2-3 days. In another embodiment of the invention, the pH of the cell culture medium is maintained within the range of about 7.2-7.6 by addition of HEPES. In a preferred embodiment, the concentration of HEPES is about 15 mM.

In another embodiment of the invention, the growth rate of the cultured cells is decreased by irradiating the cells.

In another embodiment of the invention, oocysts produced in cell culture are cryopreserved. In a preferred embodiment, the oocysts are cryopreserved in a cryopresevation solution comprising about 10% DMSO and about 90% FBS.

In one embodiment of the invention, oocysts propagated by the new cell culture methods are harvested from infected cells and used directly for experiments or cryopreserved. In a preferred embodiment, the oocysts are used to infect fresh cultured cells. In a most preferred embodiment, oocysts harvested from infected cultured cells are repeatedly passaged in fresh cultured cells to maintain an essentially continuous supply of Cryptosporidium. In one embodiment, cell culture medium is harvested from Cryptosporidium-infected cells and used directly to infect fresh cells. In another embodiment, oocysts are recovered from medium obtained from Cryptosporidium-infected cells, then recovered by centrifugation, and the resuspended oocysts are used to infect fresh cells.

The invention also includes the oocysts produced by the new propagation method and compositions thereof. In a preferred embodiment, the invention includes a composition comprising oocysts produced by the new cell culture method and a pharmaceutical carrier.

The invention further encompasses the use of Cryptosporidium produced by the new culture methodology for inducing antibodies in an animal, especially antibodies providing protective immunity. Methods for producing intestinal protozoan vaccines are described, for example, by Olson (U.S. Pat. Nos. 5,512,288 and 6,153,191; hereby incorporated by reference). The extracellular forms of Cryptosporidium of the present invention will be particularly useful in producing a vaccine that will elicit a protective immune response in animals and humans. The invention also includes a vaccine composition comprising oocysts produced by the new cell culture method. In a preferred embodiment, the composition comprises a suitable pharmaceutical carrier, which may be an adjuvant.

The invention also encompasses a method of producing antibodies to Cryptosporidium in an animal comprising administering to the animal an antibody-producing amount of the oocysts produced by the new cell culture method. In a preferred embodiment, the antibodies confer protective immunity to the animal suffering from or susceptible to a Cryptosporidium infection.

The invention further encompasses the use of cryopreserved Cryptosporidium produced by the new culture methodology for preparing a vaccine according to methods known in the art, such as those described in the Olson patents. In one embodiment of the invention, the oocysts are cryopreserved in a cryopresevation solution comprising about 10% DMSO and about 90% FBS.

In a further embodiment, the oocysts are recovered by centrifugation of the supernatant from infected cells.

### DESCRIPTION OF THE INVENTION

### A. DEFINITIONS

As used herein, the following terms have the following meanings:

Adjuvant: a vehicle used to enhance antigenicity. The use of adjuvants is well-known in the art. Adjuvants may include suspensions of minerals on which antigen may be adsorbed, such as alum, aluminum hydroxide or phosphate; water-in-oil emulsions in which antigen solution is emulsified in mineral oil, such as Freund's incomplete adjuvant; and may include additional factors, such as killed mycobacteria in Freund's complete adjuvant, to further enhance antigenicity.

Antibody: a molecule, especially a protein, that binds immunologically to a known antigen or a determinant of an antigen.

Apicomplexa: a phylum of parasitic protozoa lacking cilia and usually lacking flagella.

Bile: substance secreted by the liver and discharged into the duodenum where it aids in the emulsification of fats, increases peristalsis, and retards putrefaction. The term bile is also used to refer to powdered or dried bile. Bile also means any component of bile, such as an individual bile salt.

Cell culture: the in vitro growth or maintenance of isolated eukaryotic cells or parasitic organisms infecting such cells.

Colonization: attachment to the gut of the infected animal by the protozoan.

Cryopreservant: a substance suitable for cryopreserving cells by preventing the formation of ice crystals in an organism to be cryopreserved. Examples include dimethyl sulfoxide (DMSO), glycerol, and potassium chromate.

Cryopreservation: a process by which cells are stored at low temperatures (below −20° C.) in such a manner as to minimize cell death. Cells are typically stored in liquid nitrogen (−195° C.) in the presence of a cryopreservant such as dimethyl sulfoxide or glycerol. The presence of the cryopreservant prevents the formation of ice crystals in the cells which typically leads to cell death.

Cryptosporidium: a protozoan parasite that invades the intestinal epithelial cells of various mammals, including humans, domesticated farm animals, and poultry.

Cryptosporidium Infection: infection with Cryptosporidium. The symptoms of the infection, such as diarrhea, abdominal cramps, headache, gas, bloat, weight loss, lack of weight gain, dehydration, malaise, malabsorption, colonizing of the gut with the parasite, shedding of cysts, etc. are also included.

Cyst: the infectious form of many protozoal parasites, such as Cryptosporidium or Giardia. Cysts usually comprise a highly condensed cytoplasm and resilient cell wall. They are often shed in the feces, leading to the infection of other animals by the fecal-oral route of infection. Cysts may be viable, i.e. able to produce a trophozoite in a new host, or may be non-viable.

Effective Amount: dose required to protect an animal against infection or disease or alleviate a particular symptom of an infection or disease.

Excystation: the release of sporozoites from cysts.

Feed Conversion: measure of an animal's ability to gain weight expressed as the weight of feed required to produce a unit quantity of body weight. Intestinal diseases, including those caused by protozoa, reduce this parameter by making an animal less efficient in converting feed to body weight.

Gamont: a cell that has the potential to give rise to gametes (synonym: gametocyte).

Immune Response: development in the host of a cellular and/or antibody-mediated immune response to a composition or vaccine of interest.

Meronts (type I): a form of Cryptosporidium that comprises eight type-I merozoites.

Meronts (type II): a form of Cryptosporidium that comprises four type-II merozoites. Type II meronts are thought to be formed by type I merozoites.

Merozoites (type I): a form of Cryptosporidium resulting from multiple fission (i.e., merogony, schizogony), believed to be capable of undergoing merogony and recycling indefinitely.

Merozoites (type II): merozoites liberated from type II meronts. Type II merozoites enter host cells, enlarge, and form (i) macrogametes (i.e., a macrogametocyte) or (ii) undergo multiple fission to become microgametocytes comprising 16 flagellated microgametes. Microgametes released from ruptured microgametocytes penetrate macrogametes to form a zygote.

Neutralize: able to prevent or alleviate toxic effects.

Oocysts: also called “thick-walled” oocysts; a form of Cryptosporidium found in the environment and resistant to environmental insults; oocysts comprise four infective sporozoites resulting from meiotic division of a zygote. About 20% of oocysts fail to develop a thick wall and become “thin-walled” oocysts that may excyst in the gut and reinfect the host. Oocysts are an infective stage of Cryptosporidium.

Prevention of symptoms: includes prevention of any effect caused by Cryptosporidium or by a toxin of Cryptosporidium.

Production in vitro: production in culture, not in an infected host animal. Production in vitro includes recombinant production.

Protectively Immunogenic: able to protect an animal against infection or disease or alleviate a particular symptom of an infection or disease.

Recombinantly produced: produced by means of gene expression in any heterologous system including microorganisms, plants or animals and/or chemically synthesized by methods known in the art when the sequence is known.

Sonication: disruption of cells by exposing a suspension of the cells to high frequency sound waves.

Sporozoite: an infectious form of Cryptosporidium resulting from the meiotic division of a zygote (i.e., sporogeny).

Subunit: any part of Cryptosporidium which is less than the whole organism. Subunits that are antigenic may be used in vaccine compositions to produce an immune response. Subunits may include flagella, the ventral adhesive disk, membranes, cytoskeletal membrane proteins, cytosolic membrane proteins, toxins and any other part of an organism which may be antigenic and induce an immune response. This includes recombinantly produced subunits.

Toxin: a noxious or poisonous substance that is produced by Cryptosporidium. It may be an extracellular product (exotoxin). When intestinal cells are affected, a toxin is classified as an enterotoxin. This includes recombinantly produced toxins.

Trophozoite: the vegetative form of Cryptosporidium.

Vaccine Strain: a strain of Cryptosporidium which is protectively immunogenic when administered to an animal. This includes strains which have been genetically manipulated.

Zygote: the only diploid form in the Cryptosporidium life cycle; formed by the fusion of microgametes and macrogametes. Zygotes undergo meiosis to form four sporozoites.

### B. DETAILED DESCRIPTION OF THE INVENTION

The instant invention is drawn to an improved method of propagating Cryptosporidium, including but not limited to C. parvum and C. andersoni, in cell culture. The method is based on the diligent maintenance of the pH of the culture medium between about 7.2 and about 7.6, achieved by regularly monitoring the pH of the medium and changing the medium or adding HEPES buffer to keep the pH within the specified range. In addition to enhancing Cryptosporidium host cell invasion, these improvements also stabilize and slow the growth of the cultured host cell, preventing cell overgrowth which would adversely affect the Cryptosporidium life cycle. Host cell monolayer overgrowth may also be slowed by irradiation of the monolayers.

The culture method of the claimed invention allows the long-term propagation of Cryptosporidium in vitro and supports the complete life cycle of the parasite. The cell culture method produces all previously known forms of Cryptosporidium, including the following:

Merozoites: thread shaped with rounded anterior and posterior ends, usually identified attempting to penetrate fresh cells after about 48 hours postinfection (hpi).

Trophozoites: about 2.7×2.7 μM in diameter, round or oval, intracellular form that is a transitional stage from sporozoites and merozoites to meronts.

Meront I: about 3.75×4 μM, developing meronts with six or eight merozoites.

Meront II: about 3.1×2.8 μM, meronts with only 4 merozoites.

Microgamonts: 5.6×3.4 μM, observed containing 14-16 non-flagellated microgametes.

Macrogamonts: 4×4 μM, distinguished based on size and large peripheral nucleus.

Oocysts: 5×5 μM, only thin-walled oocysts were observed.

Sporozoites: 5.2×1.2 μM with characteristic “comma” shape, observed continuously after about 72 hpi.

Moreover, the improved cell culture method of the instant invention has allowed the identification of two novel extracellular forms of Cryptosporidium that have not previously been observed. The following extracellular forms of Cryptosporidium were observed at 72 hpi post-infection:

Stage 1: 5.7-11.5×2.2-2.8 μM, spindle-shaped forms possessing a large posterior nucleus, round posterior structure, and granules present in the cytoplasm. This form of the parasite was actively moving.

Stage 2: 3-10×1.25-3.75 μM, blunt-ended and rod-shaped but possessing no pointed end characteristic of merozoites. Varied in size due to active expansion and contraction. Possesses a mid-to-anterior nucleus.

These additional extracellular forms of the parasite will be useful in producing a more protective vaccine because they are likely to display antigens that are not found on intracellular forms Cryptosporidium. As discussed in the Background of the Invention section, above, extracellular forms of Cryptosporidium are likely to be particularly useful in eliciting an IgA response in animals. IgA response is associated with immunity and clearing of the parasite.

The invention therefore encompasses a method of preparing a Cryptosporidium vaccine by culturing Cryptosporidium in an appropriate cell line, such at HCT-8 cells, in an appropriate Cryptosporidium maintenance medium, for example, RPMI-1640 supplemented with about 1% FBS, about 0.3 g sodium bicarbonate, about 0.03 g L-glutamine, about 0.02 g bovine bile, about 0.1 g glucose, about 25 μg folic acid, about 100 μg 4-aminobenzoic acid, about 50 μg calcium pantothenate, about 875 μg ascorbic acid, about 10,000 U penicillin G (Sigma), and about 0.01 g streptomycin per 100 ml RPMI-1640 adjusted to a final pH of 7.4, at 37° C. at 5% CO₂. Newly confluent monolayers are infected by replacing the culture medium with the above oocyst-containing medium. An oocyst inoculum of about 2,000 to about 5,000 oocysts/cm² of culture area is used to infect the monolayers. The oocysts are allowed to infect the monolayers for about 80-90 minutes before the oocyst-containing medium is replaced with fresh culture medium. Culture maintenance medium is diligently maintained at a pH between about 7.2 and about 7.6 by the addition to HEPES buffer, pH about 7.4, to the medium and by changing the medium when the pH falls outside the specified range. In one example, 15 mM HEPES is added to the medium which is additionally changed every 2-3 days [Hijjawi, N. S. et al. (2001) Complete development and long-term maintenance of Cryptosporidium parvum human and cattle genotypes in cell culture. Int. J. Parasitol. 31:1048-55].

Medium is collected from 5-day-old flasks of infected cells. Oocysts and other parasite forms are recovered by centrifugation at 2000×g for 5 minutes and the pellet is resuspended in about 2 ml PBS for use in subsequent experiments.

Alternatively, parasite-containing medium can be used to infect fresh cells, thereby perpetuating the life cycle of Cryptosporidium in vitro.

The invention further provides a method of cryopreserving Cryptosporidium propagated by the improved cell culture method.

The invention also provides vaccine compositions comprising a Cryptosporidium cultured as described herein which is effectively immunogenic in animals. Various strains of Cryptosporidium may be useful in such vaccine compositions. Strains that produce a toxin(s) when cultured in vitro are most useful for producing vaccine compositions. In a preferred embodiment, the strain is selected from the group consisting of C. parvum, C. andersoni, C. muris, and C. melegridis.

The Cryptosporidium may be cultured as set forth in the Examples section, below, then harvested for use in vaccine compositions. The protozoa may be disrupted prior to use in vaccine compositions. Various methods of disruption may be used, including but not limited to sonication, osmotic pressure, freezing, exposure to detergents such as sodium dodecyl sulfate (SDS), and heating. In a preferred embodiment, sonication is used to disrupt the parasites.

In addition to disrupting the protozoa, it may be also desirable to inactivate the Cryptosporidium, or toxins or subunits, produced by the Cryptosporidium before use in vaccine compositions. Conventional techniques such as heat treatment or formalin inactivation may be used.

Vaccine compositions may comprise one or more strains of Cryptosporidium and/or one or more subunits and/or toxins of Cryptosporidium. Such subunits and/or toxins may be used in addition to whole or sonicated protozoa or may be used in cell-free vaccine compositions.

The formulation of vaccine compositions may include suitable pharmaceutical carriers, including adjuvants. The use of an adjuvant, for example, an alum-based adjuvant, such as aluminum hydroxide, is preferred. Commercially available adjuvants may also be used in vaccine composition or combined with commonly available adjuvant in vaccine compositions. For example, a preferred vaccine composition comprises aluminum hydroxide and QUILL A (Super Fos, Copenhagen, Denmark). The precise adjuvant formulation of the vaccine compositions will depend on the particular strain of Cryptosporidium, the species to be immunized, and the route of immunization. Vaccine composition formulation is well-known to those skilled in the art.

Such vaccine compositions are useful for immunizing an animal susceptible to Cryptosporidium infection, including but not limited to, bovine, ovine, caprine, equine, leporine, porcine, canine, feline, and avian species. Both domestic and wild animals may be immunized as well as food producing animals and humans.

The present invention further provides a method of preventing or treating a disease associated with Cryptosporidium infection comprising administering an effective amount of a strain of Cryptosporidium to an animal in need of such prevention or treatment. An appropriate strain may be used in a vaccine composition as previously discussed. This method is useful in, for example, dogs, cats, sheep, humans, domestic animals (especially food producing animals), avian species, and wild animals. Use in wild animals may prevent contamination of water supplies used by humans or domestic animals.

Any convenient route of inoculation may be used to deliver the vaccine composition and the route may vary depending on the animal to be treated, and other factors. Parenteral administration, such as subcutaneous, intramuscular, or intravenous administration, is preferred. Subcutaneous administration is most preferred for canine and feline species. Oral administration may also be used, including oral dosage forms which are enteric coated.

The schedule of administration may vary depending on the animal to be treated. Animals may receive a single dose, or may receive a booster dose or doses. Annual boosters may be used for continued protection. The age of the animal to be treated may also affect the route and schedule of administration. Administration is preferred at the age when maternal antibodies are no longer present and the animal is immunologically competent. These conditions occur at about 6 to 7 weeks of age in canine or feline species. Additionally, immunization of mothers to prevent infection of their offspring through passive transfer of antibodies in their milk is also contemplated. Treatment may be administered to symptomatic or asymptomatic animals, including animals or humans with chronic infection, and may be used to increase growth rate by alleviating such symptoms of infection as diarrhea. Accordingly, administration of an effective amount of a vaccine composition may increase feed conversion.

The present invention also provides antibodies to toxins of Cryptosporidium, These antibodies may be useful as an antiserum to neutralize toxins of Cryptosporidium, thereby relieving symptoms associated with these toxins. It is expected that oral administration of these antibodies, using an enteric coated dosage formulation, will be preferred.

The invention further provides Cryptosporidium that have been propagated in culture and cryopreserved. Methods of crypreservation are described in the Examples section, below.

### C. EXAMPLE OF EMBODIMENTS OF THE INVENTION

The following examples are exemplary and are not intended to limit the scope of the invention in any manner.

1. In Vitro Propagation of Cryptosporidium

a. Purification of Oocysts

Cryptosporidium oocysts were purified from the feces of infected animals by two rounds of ficoll gradient centrifugation. Ficoll gradients were created by consecutively adding to a 10-ml centrifuge tube (i) 2.5 ml of a 1% ficoll 400 (Pharmacia, Uppsala, Sweden) solution in phosphate buffered saline (PBS) containing 16% sodium diatrizoate (ICN, Aurora, Ohio), (ii) 2.5 ml of a similar 0.5% ficoll solution, and (iii) 4 ml of fecal material suspended in PBS. Oocysts were collected from the PBS/0.5% ficoll interface following centrifugation in a swinging bucket rotor at about 1,000×g for about 15 minutes at room temperature. The oocysts were twice washed in water then resuspended in cold sterile PBS comprising antibiotics (e.g., 5 mg/ml gentamycin, 4 mg/ml lincomycin, and 10 mg/ml ampicillin) [Meloni, B. P. and Thompson R. C. (1996) Simplified methods for obtaining purified oocysts from mice and for growing Cryptosporidium parvum in vitro. J. Parasitol. 82:757-62]. In an alternative, the oocysts were resuspended in cold sterile PBS comprising 10,000 U penicillin G, and 0.01 g streptomycin per 100 ml PBS.

b. Excystation of Oocysts

Excystation was performed by adding about 0.1 ml to about 1.0 ml portions of the ficoll-purified oocysts to about 9 ml sterile-filtered weak acid solution (e.g., a hydrochloric acid solution with a final pH of about 2.5) with 0.5% trypsin and incubating at 37° C. for 20 minutes with agitation. Excysted oocysts were recovered by centrifugation at 1,500×g for 4 minutes at room temperature then resuspended in, Cryptosporidium maintenance medium, for example, RPMI-1640 (Sigma, St. Louis, Mo.) supplemented with 1% FBS, 0.03 g L-glutamine, 0.3 g sodium bicarbonate, 0.02 g bovine bile, 0.1 g glucose, 25 μg folic acid, 100 μg 4-aminobenzoic acid, 50 μg calcium pantothenate, 875 μg ascorbic acid, 10,000 U penicillin G (Sigma), and 0.01 g streptomycin per 100 ml RPMI adjusted to a final pH of 7.4 [Hijjawi, N. S. et al. (2001) Complete development and long-term maintenance of Cryptosporidium parvum human and cattle genotypes in cell culture. Int. J. Parasitol. 31:1048-55].

c. Cell Culture

Growth of Cryptosporidium may be accomplished using a variety of cultured mammalian cells. In one example, human ileocecal carcinoma cells [HCT-8 (ATCC Cat. No. CCL-244)] were used to propagate Cryptosporidium. Cells were grown in 25 cm²-cell culture flasks in, for example, RPMI-1640 (Sigma, St. Louis, Mo.) supplemented with RPMI-1640 (Sigma, St. Louis, Mo.) supplemented with about 10% FBS, 0.03 g L-glutamine, 0.3 g sodium bicarbonate, 10,000 U penicillin G (Sigma), and 0.01 g streptomycin per 100 ml RPMI adjusted to a final pH of 7.4 at 37° C. at 5% CO₂. Newly confluent monolayers were infected by replacing the cell growth medium with the above oocyst-containing maintenance medium. An oocyst inoculum of about 2,000 to about 5,000 oocysts/cm² of culture area was used to infect the monolayers. The oocysts were allowed to infect the monolayers for about 80-90 minutes before the oocyst-containing medium is replaced with maintenance medium. The maintenance medium was diligently maintained at a pH between about 7.2 and about 7.6 by the addition of HEPES buffer, pH about 7.4, to the medium and by changing the medium when the pH falls outside the specified range. In one example, 15 mM HEPES was added to the maintenance medium which was additionally changed every 2-3 days [Hijjawi, N. S. et al. (2001) Complete development and long-term maintenance of Cryptosporidium parvum human and cattle genotypes in cell culture. Int. J. Parasitol. 31:1048-55].

Medium was collected from about 5-day-old flasks of infected cells. Oocysts and other parasite forms were recovered by centriftigation at 2000×g for 5 minutes and the pellet was resuspended in about 2 ml PBS for use in subsequent experiments. The oocysts may also be resuspended in PBS comprising antibiotics such as 5 mg/ml gentamycin, 4 mg/ml lincomycin, and 10 mg/ml ampicillin or 10,000 U penicillin G, and 0.01 g streptomycin per 100 ml PBS.

Alternatively, oocysts recovered from the above 5-day-old flasks were used, either before or after recovery by centrifugation and resuspension, to infect fresh monolayers of cells. This process was repeated for up to 25 days. By periodically harvesting the Cryptosporidium and infecting fresh cells, the Cryptosporidium culture can be maintained for an indefinite period of time to provide a continuous supply of parasites.

2. Cryopreservation of Cryptosporidium Oocysts Produced in Cell Culture

Cryptosporidium oocysts propagated by the methods described herein may be cryopreserved for use in future experiments, to preserve unusual parasite variants, or simply for the convenience of the practitioner. Oocysts are separated from PBS (or other resuspension medium used following harvesting) by centrifugation then resuspended in a solution suitable for cryopreservation. Cryopreservation solutions may comprise cell culture media, FBS, and a cryopreservant such as dimethyl sulfoxide (DMSO) or glycerol. A typical cryopreservation solution comprises 5-15% DMSO added to cell culture media comprising 10-20% FBS. Resuspended oocysts are placed on ice for several minute, then at about −80° C. for several hours, and then stored in liquid nitrogen. In the alternative, the resuspended oocysts are placed directly in liquid nitrogen or in a cell freezing apparatus designed to control the freezing process.

3. Preparing Whole Sonicate Vaccine

A whole sonicate vaccine of Cryptosporidium is prepared using, for example, a Virsonic Cell Disrupter while maintaining the cell-culture-derived parasite suspension on ice. Three 20 second bursts are generally sufficient to disrupt the parasites. The presence of intact trophozoites may be checked using a hemacytometer and an additional 20 second burst is used where necessary. The final protein concentration of the sonicate is determined using the BIORAD Protein Assay and adjusted to 0.75 mg/ml by the addition of sterile PBS. This solution is then mixed 1:4 with the previously described alum-based adjuvant for use as a vaccine preparation for immunizing animals in the following studies.

4. Immunizing Animals with Cryptosporidium Oocysts Produced in Cell Culture

Sonicated Cryptosporidium preparations, concentrated Cryptosporidium toxin, and other Cryptosporidium-containing preparations may be used to immunize animals against Cryptosporidium. Methods of immunizing animals against Cryptosporidium are adapted from those used to immunize against Giardia as described by Olson (U.S. Pat. Nos. 5,512,288 and 6,153,191) with minor modifications.

For example, two groups of five calves each are immunized (Group A) or mock-immunized (Group B) by subcutaneous injection with about 0.5 ml of an above-described adjuvant and about 2.5 ml an above Cryptosporidium preparation (Group A) or about 2.5 ml PBS (Group B). Animals may be checked for the presence of antibodies to Cryptosporidium antigens using an ELISA assay wherein purified Cryptosporidium antigen is immobilized on the ELISA plates. The presence of Cryptosporidium antibodies in the serum of immunized cattle indicates that a humoral immune response has produced antibodies to Cryptosporidium antigens present in the vaccine.

5. Challenging Inoculated Animals with Cryptosporidium

To determine whether these antibodies are protective against subsequent Cryptosporidium challenge, the immunized or mock immunized animals are challenged with Cryptosporidium parasites. Cryptosporidium parasites are introduced either orally or by direct intestinal inoculation. Typically, mice are challenged with about 10⁶ oocysts and calves are infected with about 10⁷ to about 10⁸ oocysts (see, e.g., Perryman, L. E. et al. (1999) Protection of calves against cryptosporidiosis with immune bovine colostrum induced by a Cryptosporidium parvum recombinant protein. Vaccine. 17:2142-49; Bukhari, Z. et al. (2000) Comparison of Cryptosporidium parvum viability and infectivity assays following ozone treatment of oocysts. Appl. Envir. Microbiol. 66:2972-80; Sréter, T. et al. (2000) Morphologic, host specificity, and molecular characterization of a Hungarian Cryptosporidium meleagridis isolate. Appl. Envir. Microbiol. 66:735-738, and references within).

6. Monitoring Animals for Clinical Evidence of Infection

Cryptosporidium-challenged animals are monitored for overt clinical signs of disease, including but not limited to soft stools, diarrhea, weight loss, lethargy, and failure to thrive. Fecal cyst counts are also performed daily for the duration of the infection to determine whether the infected animals are shedding Cryptosporidium oocysts. Serum samples are obtained at least weekly and at post mortem for use in ELISAs to measure IgM and IgG titers. Following euthanasia, gut samples (e.g., duodenum, jejunum, ileum) are taken for trophozoite counts, light microscopy, and electron microscopy. Mucosal scrapings, serum samples and bile are collected and stored frozen at about −80° C. for further immunological analyses and enzymatic investigations. Reduced clinical manifestation of Cryptosporidium infection in immunized animals, compared with control animals that are not immunized, is evidence that the vaccine is effective in protecting immunized animals against Cryptosporidium infection.

7. Enzyme Linked Immunosorbent Assay (ELISA)

Animal gut mucosal homogenates are prepared essentially as described by Olson et al. (U.S. Pat. No. 6,153,191). Tissue from the intestinal mucosa of infected animals is homogenized in 10% weight/volume 2 mM EDTA then stored at −80° C. Samples are then thawed and diluted about 1:1 with a solution comprising 2 mM EDTA, 1 and mM PMSF. The mixture is dispersed and disrupted by five passes through an 18 G needle. Insoluble debris is pelleted by centrifugation at about 17,000×g for 20 minutes. Supernatants containing soluble proteins are used for ELISAs immediately or stored at −80° C. Polyclonal or monoclonal antibodies that detect Cryptosporidium antigen are useful in the assay. Samples may be further diluted to obtain signals within the linear range of the assay. All samples are assayed in duplicate. See also, Perryman, L. E. et al. (1999) Protection of calves against cryptosporidiosis with immune bovine colostrum induced by a Cryptosporidium parvum recombinant protein. Vaccine. 17:2142-49.

The detection of antibodies to Cryptosporidium proteins in the serum of immunized animals is evidence of a humoral immune response to the vaccine. By using different purified Cryptosporidium antigens in the ELISA, one skilled in the art can also determine to which antigens the immunized host responds. These data can be correlated with the results of Cryptosporidium challenge assays to determine what antibodies afford protection against infection.

8. Phylogenetic Analysis of Cryptosporidium obtained from Challenged Animals

It may be desirable to perform genetic analysis on Cryptosporidium samples obtained from infected animals to determine whether Cryptosporidium that continue to propagate in immunized animals, if any, have accumulated genetic polymorphisms that allow the parasites to escape the host immune response. DNA is isolated from oocysts present in the feces of infected animals, or from other Cryptosporidium-containing samples, using standard molecular biology techniques well known to those skilled in the art. At least five primers that are useful in distinguishing between Cryptosporidium species are described in Morgan, U. M. et al. [(1995) Molecular characterization of Cryptosporidium isolates from humans and other animals using random amplified polymorphic DNA analysis. Am J. Trop. Med. Hyg. 52:559-64]. Products obtained from PCR using these primers can be analyzed directly by electrophoresis to determine if polymorphisms are evident. PCR products can also be sequenced using standard methods.

Modifications of the above-described modes of carrying out the various embodiments of this invention will be apparent to those skilled in the art based on the above teachings related to the disclosed invention. The above embodiments of the invention are merely exemplary and should not be construed to be in any way limiting.

The disclosure of each publication, patent, and patent application cited above is hereby incorporated by reference in its entirety.

What is claimed is:

1. A method of propagating Cryptosporidium oocysts in vitro comprising the steps of:
a) isolating Cryptosporidium from an infected host organism,
b) excysting the isolated oocysts,
c) recovering the excysted oocysts by centrifugation,
d) resuspending the recovered oocysts in cell culture medium, said medium having a pH within a range of about 7.2-7.6,
e) inoculating cultured mammalian cells with the resuspended oocysts,
f) maintaining the culture of inoculated cells within a pH range of about 7.2-7.6,
g) harvesting supernatant from the inoculated cells, and
h) recovering the oocysts produced in the inoculated cells.

2. The method of claim 1 wherein the host organism is selecting from the group consisting of a mouse, a human, a bovine, and a pig.

3. The method of claim 1 wherein the mammalian cells are irradiated.

4. The method of claim 1 wherein the Cryptosporidium is selected from the group consisting of Cryptosporidium parvum and Cryptosporidium andersoni.

5. The method of claim 1 wherein the cell culture medium comprises an antibiotic.

6. The method of claim 1 wherein the cell culture medium comprises an antifungal agent.

7. The method of claim 1 wherein the cell culture medium comprises about 0.2 g/L bile.

8. The method of claim 1 wherein the cells are human adenocarcinoma cells.

9. The method of claim 1 wherein the cells are HCT-8 cells.

10. The method of claim 1 wherein the isolated oocysts are excysted by pretreatment with a trypsin solution at low pH.

11. The method of claim 10 wherein the pH of the pretreatment solution is within a range of about 2.5 to about 3.

12. The method of claim 10 wherein the pretreatment solution comprises about 0.5% trypsin.

13. The method of claim 10 wherein the oocysts are incubated in the pretreatment solution for about 20 minutes at about 37° C.

14. The method of claim 1 wherein the cell culture medium further comprises at least one additional supplement selected from the group consisting of folic acid, 4-aminobenzoic acid, calcium pantothenate, and ascorbic acid.

15. The method of claim 1 wherein the pH of the cell culture medium is maintained within the range of about 7.2-7.6 by periodically changing the cell culture media.

16. The method of claim 1 wherein the pH of the cell culture medium is maintained within the range of about 7.2-7.6 by addition of HEPES.

17. The method of claim 16 wherein the concentration of HEPES is about 15 mM.

18. The method of claim 1 further comprising the step of cryopreserving the recovered oocysts.

19. The method of claim 18 wherein the cryopreservation is performed using a cryopreservation mixture comprising about 10% DMSO and about 90% FBS.

20. Oocysts produced by the method of claim 1.

21. A composition comprising oocysts produced by the method of claim 1.

22. A method of producing antibodies to Cryptosporidium in an animal comprising administering to the animal an antibody-producing amount of the oocysts produced by the method of claim 1.

23. The method of claim 22 wherein the antibodies confer protective immunity to the animal suffering from or susceptible to a Cryptosporidium infection.

24. A vaccine composition comprising oocysts produced by the method of claim 1 and a suitable pharmaceutical carrier.

25. The method of claim 1 wherein steps e) through h) are repeated at least once.

26. The method of claim 1 wherein the oocysts are recovered by centrifugation of the supernatant.
